(19) United States
(12) Patent Application Publication
(10) Pub. No.: US 2003/0050691 A1
(43) Pub. Date: Mar. 13, 2003

(54) Non-thrombogenic implantable devices

(76) Inventors: Edward Shifrin, Raanana (IL); Dmitri Menglet, Victoria (AU); Nikolai G. Sedelnikov, Moscow (RU); Gennady Nickelshpur, Haifa (IL)

Correspondence Address: FROMMER LAWRENCE & HAUG, 745 FIFTH AVENUE- 10TH FL., NEW YORK, NY, 10151, US

(21) Appl. No.: 10/205,349

(22) Filed: Jul. 25, 2002

Related U.S. Application Data
(63) Continuation of application No. PCT/IL01/00131, filed on Feb. 8, 2001.

(30) Foreign Application Priority Data
Feb. 9, 2000 (IL) 134452
Aug. 14, 2000 (IL) 137839

(51) Int. Cl. A61F 2/06

(52) U.S. Cl. 623/1.23; 623/1.46; 427/2.25

(57) ABSTRACT

A prosthetic material having non-thrombogenic properties for the vascular system of the body. The material has a base layer made from a suitable material, and a thin substantially amorphous or quasi-amorphous and preferably non-continuous layer made from a suitable metal covering at least part of the base layer. The metal layer is made from a suitable metal such as to provide a substantially non-positive electrode potential with respect to a bloodstream in contact with said metal layer. The metal layer forms the bloodstream facing surface of the prosthetic material, which may be adapted to provide patches, prosthetics and other components suitable for the vascular system.

### FIELD OF THE INVENTION

The present invention relates generally to prosthetic materials and more specifically to prosthetic devices in the form of patches, tubing, valves and other components which are adapted to patch or replace tissues which have at least one bloodstream facing surface. In particular, the present invention is concerned with providing such materials which prevent or at least significantly reduce the formation of thromboses on the bloodstream facing surfaces thereof.

### BACKGROUND OF THE INVENTION

Surgical repair or replacement of major blood vessels or heart valves damaged by disease or injury is a difficult and delicate process. Where the blood vessel or valve involved has been damaged or has deteriorated to the point where it cannot be repaired, it must be replaced.

With respect to blood vessels, techniques have been developed to use arteries or veins from other parts of the patient's body, or from a suitable donor, to replace the damaged or diseased body part. Accordingly, double surgical procedures are required, in one procedure a length of vessel suitable for replacing the injured or diseased portion being removed from one part of the body, or from the donor, and in the second procedure this being implanted at the site of the injury or disease.

The use of blood vessels from donors has been successfully employed, but such procedures call for the suppression of the body's normal immune system antagonism toward the presence of foreign tissue. Although this procedure has become safer and more readily regulated, it requires drugs, which may have untoward side effects on the patient. Certainly, the simpler and more straightforward a surgical procedure, the greater the likelihood that the patient will tolerate it well and will make a satisfactory recovery.

More recently, artificial shunts, grafts, patches and heart valves have been developed for replacing the body parts found to be damaged or defective, by suitable surgically implantation procedures. Such artificial components have been made from materials selected for their capacity to be tolerated well by the human body, to handle the requirements of fluid pressures demanded of the affected blood vessel or valve, and for their ability to provide attachment sites for the anchoring of sutures and the formation of scar tissue. Among such materials are polytetrafluoroethylene or PTFE (e.g. sold under the registered trademark “Teflon”) and polyethylene glycol terephthalate (e.g. sold under the registered trademark “Dacron”). Both are especially well-suited for producing knitted, woven or braided implants, grafts, or attachment cuffs Another material so used for shunts, grafts and patches is an expanded microporous polytetrafluoroethylene or ePTFE (e.g. sold under the registered trademark “Gore-Tex”). Examples of catheters, heart valves, or plastic reconstructive surgical material, to be at least partially embedded in an implantation site in soft organic tissue of a living organism are shown and described in U.S. Pat. No. 5,219,361 (von Recum and Campbell) and U.S. Pat. No. 5,011,494 (von Recum and Campbell). The soft tissue implant devices are for promoting anchorage thereof at the implantation site and the growth of collagen at the implantation site, and include a body defining a surface layer extending over the portion of the body contacting the organic tissue. The surface layer defines a three-dimensional pattern with an exterior surface defining a plurality of spaces and a plurality of solid surface portions,

The presence or formation of thromboses or blood clots is of significant concern in any surgical procedure, and is also a most serious problem in using arterial-venous shunts and grafts and patches or artificial heart valves. Clotting frequently occurs in dialysis shunts or grafts, requiring removal of the shunts or grafts, cleaning and surgical re-implantation. The formation and dislodging of a clot may result in the occlusion or blocking of a blood vessel, interrupting the life-giving flow of blood to major organs of the body. Formation of thromboses in surgically implanted arterial or venous grafts may result because of such factors as the woven, porous nature of the graft material, a construction of which may attract blood platelets or debris in the blood stream. The graft's chemical composition, its compliance, and/or its electro-negativity, each of which may evoke a different tissue reaction may also contribute to thrombosis. See, for example, Greisler, et al., “Plasma Polymerized Tetrafluoroethylene/Polyethylene Terephthalate Vascular Prostheses”, Arch. Surg. Vol. 124, pp. 967-972 (August 1989). This creates the attendant risk that once a mass of detritus reaches a significant weight and size, it may adhere to the wall of the blood vessel, progressively blocking the vessel, or it may be dislodged by the flow of blood through the blood vessel and will travel until it encounters a blood vessel having a diameter less than that of the thrombus, causing a blockage.

Various methods or features for limiting formation of thromboses in vascular shunts, grafts and artificial heart valves, have been proposed in the prior art.

Examples of vascular shunts are shown and described in U.S. Pat. No. 4,167,045 (Sawyer). Sawyer teaches a vascular shunt made from Dacron, coated with glutaraldehyde-polymerized proteins, aluminium or other substances. Sawyer also teaches that early attempts to use rigid, gold tubes as vascular shunts were unsuccessful.

U.S. Pat. No. 4,355,426 (MacGregor) describes the use of metallic porous vascular grafts for prevention of formation of thromboses by the formation of a thin layer of tissue which adheres on the porous surface of the grafts. The thin layer of tissue takes a long time to form, and in the meantime thromboses may be formed, rendering the use of the grafts as surgical prostheses extremely limited. In other attempts of limiting formation of thromboses the coating materials applied to the grafts have been utilized. U.S. Pat. No. 4,718,907 (Karwoski et al.) describes a fluorinated coating applied electrically to the surfaces of interwoven fabric tube. U.S. Pat. No. 4,265,928 (Braun) describes a thin coating of an ethylene-acrylic acid copolymer.

While the use of mainly homogeneous synthetic materials, e.g. “Teflon”, “Dacron” or “Gore-Tex” appeared to be more successful as an implant material, the porous or fibrillated structure of these materials is itself a factor causing formation of thromboses. The porous or fibrillated structure of these material serves as a trap for the debris in the blood stream, thus creating the centers of formation and propagation of thromboses. In order to limit the formation of thrombosis, it has been suggested that at least one surface of the implantable device interacting with the recipient's blood should be metallized, i.e. its pores filled with a metal, or the surface as a whole coated by a thin layer of metal.

U.S. Pat. Nos. 4,557,975 and 4,720,400 (Manisso) describe the application of coatings, including metal coatings, to synthetic non-woven fabric made from microporous polytetrafluoroethylene (ePTFE), which is characterized by having a microstructure of nodes interconnected by fibrils. Continuous interporous metal coatings are provided which encapsulate at least some of the nodes and fibrils of the PTFE while maintaining substantial porosity of the material. A method is described of producing temporary liquid-filled hydrophilic microporous article resulting in an improved metal plating manufacturing process. The encapsulation of the nodes and fibrils is achieved by immersion into a liquid solution and chemical deposition of metal from that liquid. While many uses for the metal coated PTFE micro-structure are enumerated, there is no disclosure or suggestion that such a material is or may be suitable for use in preventing or reducing thromboses. Furthermore, the manufacturing process for such a material as described would result in a great deal of impurities being present in the crystalline metal layer, with unpredictable results in terms of thromboses.

U.S. Pat. Nos. 5,464,438 and 5,207,706 (Menaker) describe implantable vascular prostheses such as grafts, shunts, patches or valves, formed of synthetic, woven fibers are coated with a thin layer of metallic gold to form a non-thrombogenic surface. Methods of manufacture are also disclosed. The coating is applied to the inner wall by vapor deposition or sputtering to coat the fibers without blocking or bridging the interstices formed by the intersection of the fibers. These prostheses use the therapeutic properties of gold, together with the body's long-term tolerance to the presence of gold. The gold is applied as a continuous layer over the fibers of the patch, but leaving the original interstices of the woven material substantially intact such as to enable body tissue to infiltrate the implant and hold it firmly in place.

While such cardiovascular prostheses may serve to prevent bacterial infection, they do not provide a suitable non-thrombogenic surface for permanent implantation, since the standard electrode potential of gold is highly positive, and this encourages adsorption of blood elements onto the surface leading to thromboses.

U.S. Pat. Nos. 4,871,366 and 4,846,834 (von Recum and Cooke) describe a soft tissue implant comprising a flexible main body portion, having tissue-facing surfaces, i.e. the surfaces that face tissues such as blood vessel wall and are thus facing away from the bloodstream, and a thin layer of pure titanium covering the tissue-facing surfaces. This invention includes also a method of promoting tissue adhesion of a soft tissue host to the tissue-facing surfaces of a soft tissue implant comprising a strip of polyethylene terephthalate velour comprises the steps of cleaning the strip with a low-residue detergent and rinsing same with fresh distilled water; refluxing the strip in distilled water for one hour at a temperature of less than 30.degree C.; drying the strip in a room-temperature desicator for several days; sterilizing the strip and packaging same; degasing the strip and storing same in a dust-free environment; removing the strip from the packaging and mounting the strip in the vacuum evaporator at an approximate angle of incidence of 90.degrees C. from a pure titanium metal evaporant; evacuating the vacuum evaporator to a vacuum of about 2.times.10.sup. −5 Torr; evaporating the titanium by direct resistance heating same; coating the strip with a layer of pure titanium on the order of one micron thick; and resterilizing and implanting the titanium-coated strip into the tissue host.

However, the titanium coating, which is about 1 micron thick and continuous over the exposed surfaces of the material, is provided with the objective of promoting tissue adhesion of the soft tissue host to the tissue-facing surfaces of a soft tissue implants. Thus these patents actually teach away from using titanium films for preventing adhesion of body tissue thereon, and therefore for limiting or preventing the formation of thrombosis. In addition, the use of vacuum evaporation method described therein does not permit to control either energy or flux of the high-energy metal particles during the coating process. This in turn does not allow to control the thickness or characteristics of the metal coating, which is continuous in the microscale and mainly crystalline, nor of providing strong adhesion of the coating to the implant's surface particularly when subject to mechanical stretching or bending.

Thus, implantable devices of the prior art that have at least one surface exposed to the recipient's bloodstream are generally prone to the formation of thromboses, and the addition of a protective layer to the devices has been suggested to reduce the formation of thromboses. However, one of the causes of formation of thromboses in implantable devices is the presence of electrostatic charges on the surface exposed to the recipient's bloodstream. These electrostatic charges facilitate adsorption of blood elements onto the surface exposed to the bloodstream, which, in turn, causes formation of thromboses. The use of the protective thin metal layer made of metals with positive electrode potential, e.g. platinum, gold, etc., as used in prior art devices causes massive adsorption of negatively charged blood elements onto the surface coated by such metal layer. Therefore, such metals are unsuitable as a non-thrombogenic protective layer. On the other hand, such metals as titanium, zirconium, hafnium, vanadium, niobium, tantalum, chromium, molybdenum or tungsten have negative electrode potential and, therefore, are more suitable for this purpose. However, upon partial oxidation of the surface of these metals the resulting electrode potential may become positive. For example, titanium's standard electrode potential is about −1.6 Volts; however after spontaneous passivation of its surface (i.e. its partial oxidation by air or on its initial exposure to the bloodstream) it changes to about +0.3 Volts, thereby encouraging the adsorption of negatively charged blood elements onto the metal layer. Thus metal coated prosthetic materials taught by the prior art, even when using metals such as titanium, do not provide non-thrombogenic properties, and in fact enable the formation of thromboses.

While U.S. Pat. No. 3,914,802 describes a material having a bloodstream facing non-metallic lining comprising negatively charged silica particles, which would appear to defy the fundamental macro electroneutrality principle. It is unclear how such a lining may be created in practice as free electric charges do not exist under ambient conditions, more so under in vivo conditions. In any case, there is no disclosure or suggestion of using a metal coating over a suitable substrate to reduce thrombosis.

An aim of the present invention is to provide non-thrombogenic prosthetic material having a metal coating on the surface thereof that is in contact with the stream of blood and overcomes the disadvantages of prior art materials.

It is another aim of the present invention is to provide non-thrombogenic implantable cardiovascular devices with a metal coating on the surface thereof that is in contact with the stream of blood.

It is another aim of the present invention to combine highly non-thrombogenic properties with elasticity and high durable surface adhesion particularly with mechanical stretching or bending of such devices or material.

It is another aim of the present invention to provide a method for the manufacture of such prosthetic material in any suitable shape or form, in particular where the characteristics of the metal layer may be finely controlled.

Other purposes and advantages of the invention will appear as the description proceeds.

### SUMMARY OF THE INVENTION

The present invention relates to a non-thrombogenic prosthetic material for the vascular system of the body having at least one bloodstream facing surface, comprising a base layer made from a suitable material, and a thin substantially amorphous or at least partially amorphous layer of a suitable metal covering at least part of said base layer, said metal layer comprising said at least one bloodstream facing surface, wherein said metal layer is made from a suitable metal such as to provide a substantially non-positive electrode potential with respect to a bloodstream in contact with said metal layer.

The material may be in the form of a device adapted for implantation in the body. Thus, the present invention is thus also directed to a non-thrombogenic implantable device for the vascular system of the body having at least one bloodstream facing surface, comprising a base layer made from a suitable material, and a thin substantially amorphous or at least partially amorphous layer of a suitable metal covering at least part of said base layer, said metal layer comprising said at least one bloodstream facing surface, wherein said metal layer is made from a suitable metal such as to provide a substantially non-positive electrode potential with respect to a bloodstream in contact with said metal layer.

Such a device may be in the form of a patch adapted for grafting onto a predetermined part of the vascular system. Alternatively, such a device may be in the form of a prosthesis adapted for suitable implantation in the vascular system of the body. The prosthesis may be in any suitable form, for example in the form of a vascular graft or shunt, or in tubular form, having an inner substantially cylindrical bloodstream facing surface, said metal layer comprising said bloodstream facing surface. Alternatively, the prosthesis may comprise said material in the form of at least one component of a suitable artificial heart valve, said at least one component thereof having at least one bloodstream facing surface, said thin metal layer comprising said at least one bloodstream facing surface. Alternatively, the prosthesis may comprise said material in the form of at least one component of a suitable artificial heart assembly, said at least one component having at least one bloodstream facing surface, said thin metal layer comprising said at least one bloodstream facing surface.

Preferably, the metal layer is substantially non-continuous, and the metal layer is made from a metal having a substantially non-positive standard electrode potential. Typically, the metal layer comprises a thickness which may vary from between about 0 nm and about 400 nm, with an average thickness of between 50 nm to between about 300 nm., and preferably about 200 nm. The metal layer may be made from any one of titanium, zirconium, hafnium, vanadium, niobium, tantalum, chromium, molybdenum or tungsten, or any suitable alloy comprising at least one of titanium, zirconium, hafnium, vanadium, niobium, tantalum, chromium, molybdenum or tungsten. Typically, the metal layer comprises an oxide of any one of titanium, zirconium, hafnium, vanadium, niobium, tantalum, chromium, molybdenum or tungsten.

The prosthetic material may further comprise at least one body-tissue facing surface adapted for implantation in a body tissue

Preferably, the base layer is made from a substantially homogenous suitable synthetic material. Typically, the base layer is made from a synthetic material chosen from polyurethane, including different co-polymers thereof and polyurethane-derived materials, polytetrafluoroethylene, polyethylene glycol terephthalate, or expanded microporous polytetrafluoroethylene, and other suitable polymeric materials.

Typically, the base layer is covered by said metal layer by means of a magnetron sputtering based procedure.

The present invention is also directed to a method for providing a non-thrombogenic material for the vascular system of the body having at least one bloodstream facing surface, comprising covering at least a portion of a base layer made from a suitable material with a thin substantially amorphous or at least partially amorphous layer of a suitable metal, said at least one bloodstream facing surface of the non-thrombogenic material being comprised on said thin metal layer, wherein said metal layer is made from a suitable metal such as to provide a substantially non-positive electrode potential with respect to a bloodstream in contact with said metal layer.

Preferably, but not necessarily, the metal layer is applied non-continuously over said base layer, and the base layer may be covered by said metal layer by means of a magnetron sputtering based procedure. Such a magnetron sputtering based procedure may comprise the following steps:

(a) providing said base layer made from a suitable material and in a suitable form;

(b) placing said base layer in a vacuum chamber comprising suitable magnetron sputtering means;

(c) providing a target made from said suitable metal in said vacuum chamber;

(d) evacuating the chamber to a residual pressure;

(e) providing an atmosphere of plasma forming gas in said vacuum chamber;

(f) initiating a suitable electrical glow discharge in said vacuum chamber to provide plasma ions from said plasma forming gas directed at said metal target;

(g) sputtering metal from said metal target onto said base layer responsive to interaction of said plasma ions onto said metal target whereby to cover said base layer with a thin substantially non-continuous layer of said metal.

Optionally, the method may further comprise the following step between steps (a) and (b):

(h) cleansing said base layer using any suitable cleansing method;

Preferably, cleansing method is an ultrasonic-based cleansing method. Optionally, the method may further comprise the following step between steps (e) and (f):

(i) ionically etching at least one outer surface of said base layer;

Typically, the plasma forming gas is argon; the ionic etching step is performed at a pressure of between about 0.1 Pa to about 1.0 Pa, and preferably between about 0.3 Pa to about 1.0 Pa; a power density associated with said magnetically sputtering step is between about 4.0 W/cm² to about 6.0 W/cm^{2,} a potential associated with said magnetically sputtering step is between about 200V and between 500V.

Typically, the magnetically sputtering step is performed until an average thickness associated with said substantially non-continuous metal layer reaches between about 50 nm to between about 300 nm.

In the method, the base layer is typically made from a suitable synthetic material, in particular, a suitable synthetic material chosen from polyurethane, including different co-polymers thereof and polyurethane-derived materials, polytetrafluoroethylene, polyethylene glycol terephthalate, or expanded microporous polytetrafluoroethylene, and other suitable polymeric materials. The metal target may comprise a metal chosen from among any one of titanium, zirconium, hafnium, vanadium, niobium, tantalum, chromium, molybdenum or tungsten, or an alloy comprising at least one of titanium, zirconium, hafnium, vanadium, niobium, tantalum, chromium, molybdenum or tungsten.

Typically, the method further comprises the step of oxidising at least a portion of said substantially amorphous metal layer, optionally by exposing said metal layer to the atmosphere or exposing said metal layer to a bloodstream

Optionally, the base layer may be provided in the form of a sheet particularly adapted for providing a vascular patch, wherein said metal layer is provided on the bloodstream facing layer of said sheet. Alternatively, the base layer may be provided in the form of a tube, said method further comprising the steps:

(j) inverting the tube inside out so that the inner cylindrical surface is now outermost;

(k) re-inverting the tube so that the said inner cylindrical surface in innermost again;

wherein step (j) is performed before step (b), and step (k) is performed after step (g), whereby said metal layer is provided on said inner cylindrical surface of said tube.

Alternatively, the base layer may be provided in the form of at least one component of a suitable artificial heart valve, wherein said thin metal layer is provided on the bloodstream facing layers of said at least one component of said suitable artificial heart valve. Alternatively, the base layer may be provided in the form of at least one component of a suitable artificial heart assembly, wherein said thin metal layer is provided on the bloodstream facing layers of said at least one component of said suitable artificial heart assembly.

The present invention is also directed to a method for replacing a vascular tissue with a non-thrombogenic implant comprising the steps of:

surgically removing said vascular tissue;

surgically implanting a suitable non-thrombogenic implantable device according to the present invention.

The present invention is also directed to a method for repairing a vascular tissue with a non-thrombogenic implant comprising the steps of:

surgically preparing a damaged part of said vascular tissue to receive an implant;

surgically implanting a suitable non-thrombogenic implantable device according to the present invention on said damaged part of said vascular tissue.

### BRIEF DESCRIPTION OF THE FIGURES

FIG. 1 illustrates, in perspective view, the main elements of representative cutting of a first embodiment of the present invention.

FIG. 2 illustrates, in perspective and partial cross-sectional view, the main elements of representative cutting of a second embodiment of the present invention.

FIG. 3 illustrates, in perspective view, a part of the embodiment of FIG. 2 in detail.

FIG. 4 illustrates use of the embodiment of FIG. 2 in coronary bypass applications.

FIG. 5 illustrates, in transverse cross-sectional view, the main elements of a third embodiment of the present invention.

FIG. 6 shows the main elements of a magnetron apparatus used for providing a thin substantially non-continuous layer of metal on a substrate, according to the present invention.

FIG. 7 illustrates, in transverse cross-sectional view, details of the sputtering station of FIG. 6.

FIG. 8 shows a microscope slide image at a magnification of about 80 of an experimental specimen of a graft according to the present invention, after implantation in a test animal.

FIG. 9 shows a microscope slide image at a magnification of about 250 of the experimental specimen of FIG. 8.

### DESCRIPTION

The present invention is defined by the claims, the contents of which are to be read as included within the disclosure of the specification, and will now be described by way of example with reference to the accompanying Figures.

The present invention relates to a non-thrombogenic prosthetic material for the vascular system of the body having at least one bloodstream facing surface, in which the material may be used to provide non-thrombogenic vascular implantable devices, including prosthetics, for many applications, including but not limited to, patches, vascular grafts, components of artificial heart valves and artificial heart assemblies, and the like. The prosthetic material comprises a thin layer of a suitable metal covering at least part of a base layer made from a different material, wherein the metal layer comprises the bloodstream facing surface of the prosthetic material, the present invention is characterised in that the thin metal layer is substantially amorphous or at least partially amorphous and made of a metal such as to provide a substantially non-positive electrode potential with respect to a blood stream in contact therewith, leading to substantial non-thrombogenic and even anti-thrombogenic properties of the prosthetic material. Preferably, the metal layer is arranged in a non-continuous manner over the base layer, as will be described in greater detail hereinbelow.

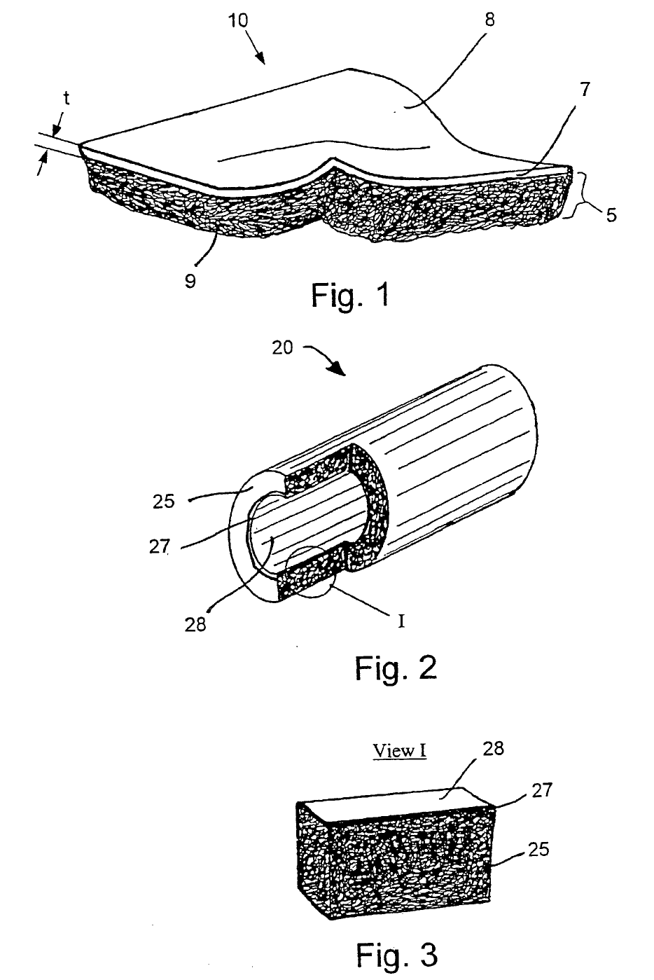

Thus, referring to FIG. 1, a first embodiment of the prosthetic material according to the present invention is in sheet form (10) and comprises a base layer (5), and a thin substantially amorphous layer (7) made from a suitable metal covering at least part of said base layer (5), typically on only one of the sides thereof, although in other applications, the base layer (5) may be covered with a metal layer (7) on either side thereof. As in all other embodiments of the present invention, the said thin substantially amorphous or at least partially amorphous metal layer (7) comprises the bloodstream facing surface (8) of the prosthetic material. Advantageously, the base layer (5) is adapted for promoting adhesion to body tissue and thus comprises a tissue facing surface (9) adapted for implantation in a body tissue. In particular, the base layer (5) itself may be adapted for implantation in a body tissue. The term “body tissue” is herein taken to include any appropriate tissue of the body with the exclusion of blood itself. The tissue facing surface (9), and In general the said base layer (5) itself, is thus typically made from a substantially homogenous suitable synthetic material, in particular including a synthetic material chosen from polyurethane, including different co-polymers thereof and polyurethane-derived materials, polytetrafluoroethylene, polyethylene glycol terephthalate, or expanded microporous polytetrafluoroethylene, and other suitable polymeric materials.

The said thin substantially amorphous metal layer (7) is made from a metal, i.e., a base metal or alloy, having a substantially non-positive, i.e., nominally zero or preferably negative, electrode potential when exposed to and in contact with a bloodstream. The metal itself from which the layer (7) is made may have a negative or even a slightly positive standard electrode potential, such that when formed as a thin substantially amorphous layer on a suitable substrate provides a substantially non-positive potential with respect to a bloodstream in contact therewith. The actual thickness (t) of the said substantially non-continuous metal layer may vary from between about 0 nm and about 400 nm, and the average thickness may vary between 50 nm to between about 300 nm., being preferably about 200 nm. Thus, the metal layer (7) is typically substantially amorphous, or quasi-amorphous, preferably comprising a substantially non-continuous structure in the micro scale, though it may nonetheless appear as a continuous layer in the macro-scale, i.e., as viewed by a user thereof. In the micro-scale, then, herein loosely defined as a microscopic scale with a resolution of about 1 to 10 nm, the metal layer (7) may comprise a plurality of surface discontinuities or openings which are substantially devoid of any metal thereon and typically exposing parts of the base layer (5) at these openings. Alternatively, or indeed at other parts of the metal layer (7), the metal layer (7) may comprise specific depositions of metal which are disconnected one from the other, forming an “island” type pattern in the micro scale. Alternatively, or indeed at other parts of the metal layer (7), the metal layer (7) may be in the form of a net-like structure wherein some areas of metal deposition may be intercalated with voids, but nonetheless fully or partially connected one to another. Thus the term “non-continuous” in the context of the metal layer (7) according to present invention is to be understood as relating to the micro-scale structure thereof, as described more fully hereinbefore, so that at least part of the material of the base layer (5) may be exposed to the bloodstream via small voids or the like formed in the metal layer (7) the exposed areas being arranged in a statistically even manner with respect to the metal layer (7).

Typically, about 50% to about 95% of the surface of the base layer (5) is actually covered by metal of the said non-continuous metal layer (7).

The metal layer (7) according to the present invention is thus different in form and function to the metal coatings of implantable devices known in the art. As explained earlier, implantable devices generally have at least one surface exposed to the recipient's bloodstream. If this surface does not have a protective layer it often causes formation of thrombosis. Known attempts to prevent formation of thromboses rely on the formation of a tissue layer over a metal coating that is applied to the device. However, these formation of such tissue layer is generally too slow to compete with the formation of thromboses, and such devices are generally unsuccessful. One of the causes of formation of thromboses in implantable devices is the presence of electrostatic charges on the surface exposed to the recipient's bloodstream. These electrostatic charges facilitate adsorption of blood elements onto the surface exposed to the bloodstream, which, in turn, causes formation of thromboses. The use of the protective thin metal layer made of metals with positive electrode potential, e.g. platinum, gold, etc., as used in prior art devices causes massive adsorption of negatively charged blood elements onto the surface coated by such metal layer. On the other hand, such metals as titanium, zirconium, hafnium, vanadium, niobium, tantalum, chromium, molybdenum or tungsten have negative electrode potential and, therefore, are more suitable for this purpose. However, upon partial oxidation of the surface of these metals the resulting electrode potential may become positive. For example, titanium's standard electrode potential is about −1.6 Volts; however after spontaneous passivation of its surface (i.e. its partial oxidation by air or on its initial exposure to the bloodstream) it changes to about +0.3 Volts, thereby encouraging the adsorption of negatively charged blood elements onto the metal layer. Thus metal coated prosthetic materials taught by the prior art, even when using metals such as titanium, do not provide non-thrombogenic properties, and in fact enable the formation of thromboses. In sharp contrast to prior art prosthetic material used for implants, for example, the micro-scale structure of the metal layer (7) according to the present invention, in particular its substantially amorphous or quasi-amorphous metal structure in particular together with its relatively low, and preferably non-regular, thickness, enables the metal film or coating to retain an almost zero or even slightly negative electrode potential on exposure to the bloodstream, or to air, water etc. Accordingly, such a substantially amorphous thin metal layer (7) provides non-thrombogenic or even anti-thrombogenic properties, since blood elements are either not encouraged to adsorb onto the surface of, or indeed are actively repelled from, the metal layer (7).

Unlike the crystalline state, the amorphous state is characterized by comprising a liquid-like structure, i.e. one lacking the long-range order. Thus the energy of chemical bonding between the atoms comprising an amorphous solid may differ drastically from that in a crystal of the same chemical substance. Therefore, both chemical and physical properties, such as reactivity and electrode potential, may vary depending on whether the substance is in an amorphous or crystalline state, i.e. its degree of crystallinity.

The terms “quasi-amorphous” and “at least partially amorphous” are used interchangeably herein to mean that the structure of the substance in question comprises in the main, or in a significant portion thereof, the amorphous phase, and may in addition also comprise in part the crystalline phase.

As described above, the electrode potential of a metal depends on the degree of crystallinity, and the electrode potential of an amorphous or quasi-amorphous metal will normally tend to be more negative than that of the same metal in the crystalline phase. Thus by forming a film or coating of amorphous or quasi-amorphous metal over a suitable substrate it is possible to “tune” the electrode potential of the metal coated material in such a way that is becomes substantially non-positive and, therefore, non-thrombogenic, when exposed to a bloodstream, while the same metal coating in the crystalline phase could have a positive electrode potential in relation to a bloodstream. In this respect, the thickness and topography of the metal coating allows some control over the relative proportion of the amorphous phase relative to the crystalline phase, as relatively thick metal coatings tend to be increasingly crystalline. The method of deposition, in particular the energy of deposition and the ambient pressure under which the deposition is conducted, are important factors in determining the relative proportion of amorphous phase in the metal layer. Also, the choice of the metal itself is very important as some metals, such as gold for example, will still provide a high positive potential, even when in the amorphous state, and are thus not suitable.

Thus, the metal layer (7) is advantageously made from any suitable metal in particular chosen from any one of titanium, zirconium, hafnium, vanadium, niobium, tantalum, chromium, molybdenum or tungsten, or indeed any alloy comprising any one of these metals, including nitrides and carbides. Further, the metal layer (7) typically comprises a corresponding oxide of any one of titanium, zirconium, hafnium, vanadium, niobium, tantalum, chromium, molybdenum or tungsten, respectively, typically formed naturally by the exposure of the corresponding metal layer (7) to air or a bloodstream.

The prosthetic material according to the first embodiment of the present invention, being in the form of a sheet (10), may be thus be provided in any suitable size and shape according to need, or may be cut to size and shape from standard sized sheets. Accordingly, an implantable device may be made from such a sheet (10) formed or cut to the appropriate dimensions and thus adapted for grafting onto a predetermined part of the vascular system of the body.

As will be further understood with reference to other embodiments of the present invention described hereinbelow, the small thickness and non-continuous characteristics of the metal layer (7) have other important advantages. In particular, such a structure for the metal layer (7) enables the prosthetic material to flexed and/or stretched to greater extents than is possible with truly continuous metal coatings of the prior art, enabling the prosthetic material of the present invention to be used for many applications not hitherto possible or practical.

Thus, referring to FIGS. 2 and 3, in a second embodiment of the present invention said non-thrombogenic prosthetic material is provided in the form of a prosthesis adapted for suitable implantation in the vascular system of the body, said prosthesis being exemplified as a vascular shunt or graft (20).

The said vascular graft (20) comprises said prosthetic material as described with respect to the first embodiment, mutatis mutandis, but in tubular form rather than in sheet form. Thus, the vascular graft (20) of the second embodiment comprises an inner, substantially cylindrical bloodstream facing surface (28), comprised on a thin at least partially amorphous and preferably non-continuous metal layer (27) that coats an outer cylindrical base layer (25), similar to the bloodstream facing surface (8), metal layer (7) and base layer (5), respectively, described for the first embodiment, mutatis mutandis. The vascular graft (20) may be optionally coated or covered by a outer layer of suitable material (not shown) for protection thereof against mechanical shocks, and/or for preventing leaks therefrom.

The flexural properties provided by the thin substantially amorphous metal layer (27) enable the vascular graft (20) to be formed by inverting the base layer (25) inside out so that the originally innermost inner cylindrical surface is now outermost, coating this surface with a thin metal layer (27), and then re-inverting the base layer (27) to reposition the inner cylindrical surface inwardly once again. With thicker or fully continuous metal layers, such inversions cannot be performed at all once the metal layer has been formed, or at best results in poor adhesion of the metal layer on the base layer, and/or cracking of the metal layer.

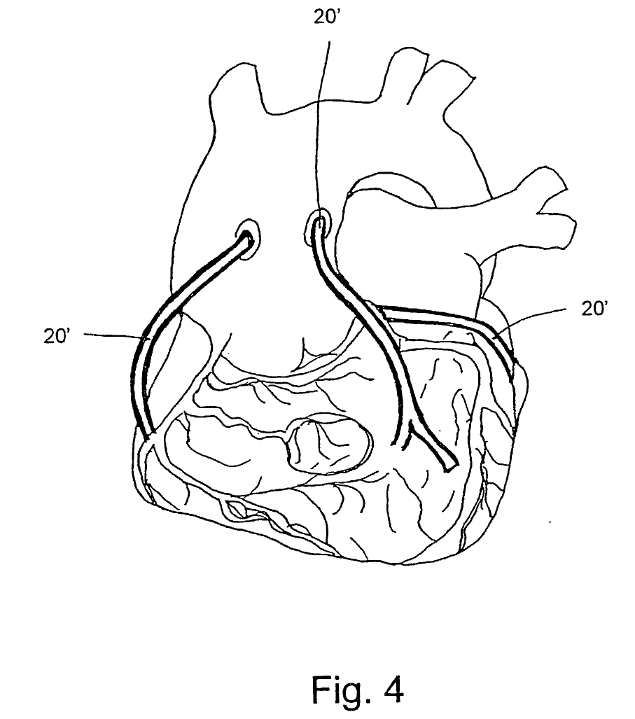

As illustrated in FIG. 4, for example, such vascular grafts (20) may be used, for example, as coronary artery bypass prostheses (20′), or indeed as prostheses for replacing varicose veins, and so on.

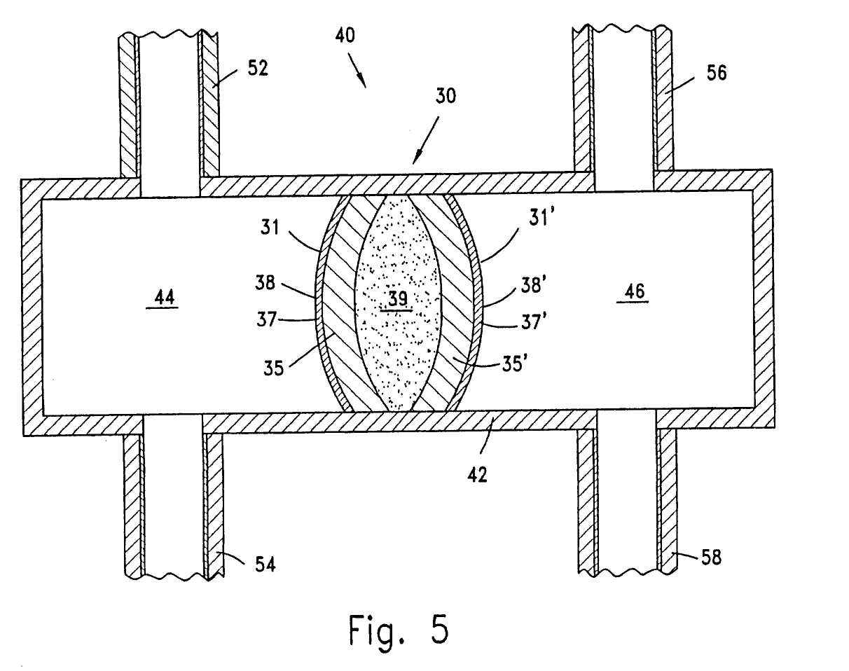

Referring to FIG. 5, in a third embodiment of the present invention said non-thrombogenic prosthetic material is provided in the form at least one component of a suitable artificial heart valve assembly (40), said component being exemplified as a flexible valve member (30).

The artificial heart assembly (40) comprises a substantially rigid valve body (42), typically made from a metal such as titanium, defining two chambers (44) and (46) therein separated by said valve member (30). Tubulures (52) and (54) provide inlet and outlet ports to the first chamber (44), and tubulures (56) and (58) provide inlet and outlet ports to the second chamber (46). The said valve member (30) comprises said prosthetic material as described with respect to the first embodiment, mutatis mutandis, but in the form of a pair of discs of said prosthetic material arranged back-to-back rather than in sheet form. Thus, the valve member (30) of the third embodiment comprises a first disc (31) having a bloodstream facing surface (38), comprised on a thin substantially non-continuous metal layer (37) that coats an inner disc-like base layer (35), similar to the bloodstream facing surface (8), metal layer (7) and base layer (5), respectively, described for the first embodiment, mutatis mutandis. The valve member (30) also comprises a second disc (31′) having a bloodstream facing surface (38′), comprised on a thin substantially non-continuous metal layer (37′) that coats an inner disc-like base layer (35′), similar to the bloodstream facing surface (8), metal layer (7) and base layer (5), respectively, described for the first embodiment, mutatis mutandis. The first disc (31) and the second disc (31′) are arranged back to back such that the base layers (35) and (35′) are facing one another and form a cavity (39) between them which is filled by a suitable gel sealed therein.

Thus, one bloodstream facing surface (38) is in communication with the first chamber (44), and the other bloodstream facing surface (38′) of the valve member (30) is in communication with the second chamber (46).

The flexural properties provided by the thin metal layers (37) and (37′) according to the present invention enable the valve member (30) to be continually flexed in normal operation of the artificial heart assembly (40) without the need for the application of large actuation forces, or without damaging these metal layers. With thicker metal layers, such continual flexing cannot be performed except with the application of relatively large actuating forces to overcome the flexural rigidity thereof, and also results in poor adhesion of the metal layer on the base layer, and/or cracking of the metal layer.

Similarly, other components of the artificial heart assembly (40), including, for example, the tubulures (52), (54), (56) and (58) may also be made as prostheses formed from said prosthetic material according to the present invention in a similar manner as described above, mutatis mutandis.

The present invention also relates to a method for providing a non-thrombogenic material for the vascular system of the body having at least one bloodstream facing surface, comprising covering at least a portion of a base layer made from a suitable material with a thin substantially non-continuous layer of a suitable metal, said at least one bloodstream facing surface of the non-thrombogenic material being comprised on said thin substantially non-continuous layer of a suitable metal.

The most successful methods for providing thin metal layers are vacuum deposition techniques, in particular: vacuum evaporation, plasma spraying and magnetron sputtering methods.

The vacuum evaporation method includes three main stages, evacuating the working chamber, evaporation (e.g. from a crucible) of preheated material used for coating and condensation of the vapor on a substrate. Despite its simplicity and the ability to provide high rates of deposition this method does not produce highly adhesive metal coatings or layers, and cannot be used successfully for metals with high melting points, such as tantalum, niobium and zirconium. Further, ejection of large metal particles in the form of droplets of molten metal is possible during evaporation, which is undesirable.

The electron-beam vacuum evaporation method is suitable for metals with high-melting points and has high deposition rates (up to 50 nanometers per second). But this method has very low efficiency (only 1-5% of all energy is used for evaporation). The substrate is exposed to high temperatures, which may cause distortion, degradation or even destruction thereof. Ejection of molten metal droplets is also possible, which is undesirable.

The plasma spraying method enables highly adhesive films of almost any metals to be formed on substrates without exposing the latter to high temperatures. This method comprises the steps of evaporation of the desired metal, ionization of the metal vapor to form a plasma flux and condensation of the metal on the substrate from the plasma flux. Concentration of the metal ions in the flux may be up to 90%, while their average energy is 50-70 electron-volt, which provide conditions for good adhesion by the metal onto the substrate. Unfortunately, it is possible to have local overheating of the substrate. Ejection of large (10-15 micrometers) metal particles as droplets of molten metal is also possible during evaporation, which as with other methods described above is undesirable.

The preferred method according to the present invention for covering said base by said thin substantially non-continuous metal layer is by means of a magnetron sputtering based procedure. The magnetron sputtering method for forming a thin, preferably non-continuous, amorphous metal layer on a suitable substrate has a number of advantages: high adhesiveness of the resulting metal layer on the substrate; the adaptability of the method for use with any metal, including metals with high melting points, absence of ejection of molten metal droplets, possibility of applying the sputtering method to assemblies or to production lines which are used in a continuous manner.

The magnetron ionic sputtering method is based on such physical phenomena as: ionization of a plasma forming gas, electrical glow discharge in vacuum, and sputtering of the metal target's material by bombarding it with accelerated ions.

The preferred method, according to the present invention, is directed to the formation of a thin substantially amorphous, and preferably non-continuous, layer of a suitable metal over a suitable base layer such as to create a substantially non-positive electrode potential with respect to a bloodstream, to provide a non-thrombogenic prosthetic material. In particular, the preferred embodiment of method of the present invention is a magnetron sputtering based procedure comprising the following steps:

(a) providing said base layer made from a suitable material and in a suitable form;

(b) cleansing said base layer, preferably ultrasonically;

(c) placing said base layer in a vacuum chamber comprising suitable magnetron sputtering means;

(d) providing a target made from said suitable metal in said vacuum chamber;

(e) evacuating the chamber to a residual pressure;

(f) providing an atmosphere of plasma forming gas in said vacuum chamber;

(g) ionically etching at least one outer surface of said base layer;

(h) initiating a suitable electrical glow discharge in said vacuum chamber to provide plasma ions from said plasma forming gas directed at said metal target;

(i) sputterings metal from said metal target onto said base layer responsive to interaction of said plasma ions onto said metal target whereby to cover said base layer with a thin substantially amorphous and preferably non-continuous layer of said metal.

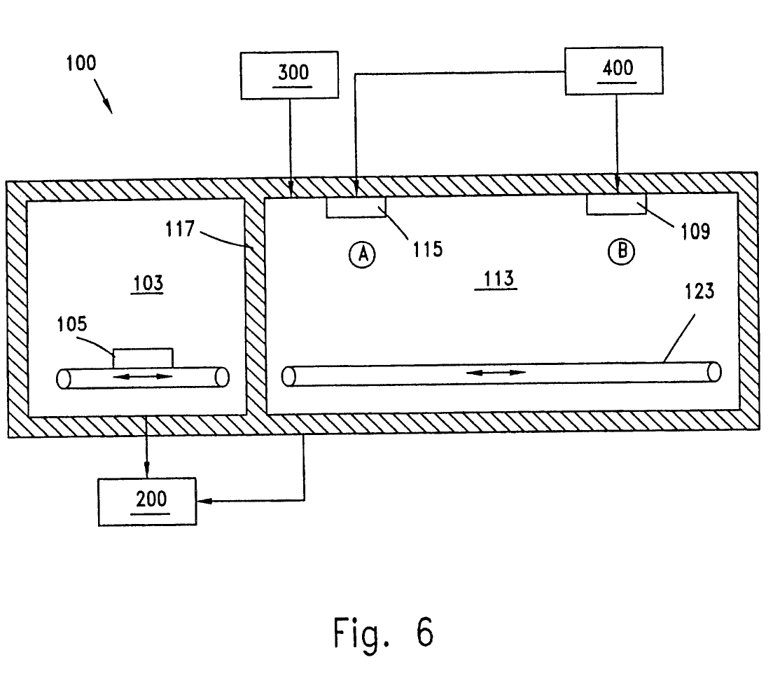

Thus, referring to FIG. 6, the magnetic sputtering apparatus, generally designated at (100), comprises a air-lock (103) in communication with a working chamber (113) via a sealable opening (117). The air-lock (103) comprises a substrate holder (105) for supporting a base layer (135) during manufacture of the prosthetic material according to the invention. The working chamber (113) comprises suitable sputtering means for providing a glow discharge next to a suitable metal target in a rarefied plasma forming gas, as will be described in greater detail hereinbelow. A conveyor system (123) or any other suitable transportation system is provided for transporting the base layer (135), supported by the substrate holder (105), from the air-lock (103) into the different stations (A) and (B) within the working chamber (113) as required, and for removing the prosthetic material formed thereon to the air-lock (103). A suitable sealable opening (not shown) enables the air-lock (103) to be accessed from the outside of the apparatus (100), in particular to enable workpieces to be inserted into the air-lock (103) and finished products removed therefrom. A second air-lock (not shown) may be provided downstream of the sputtering station (B) to facilitate the use of the apparatus (100) for mass production, by enabling finished products to be removed from the working chamber (113) without interfering with the introduction of new work pieces thereinto via air-lock (103). The sputtering apparatus (100) is serviced by an evacuation system (200) for providing a vacuum or near vacuum in the air-lock (103) and in the working chamber (113), and by a suitable gas supply (300) for supplying argon gas, or any other suitable plasma forming gas, into the working chamber (113). A suitable power supply (400) provides power to an ionisation source (115) situated at the ionic etching position (A) in the working chamber (113), and to a magnetron assembly (109) situated at the sputtering position (B).

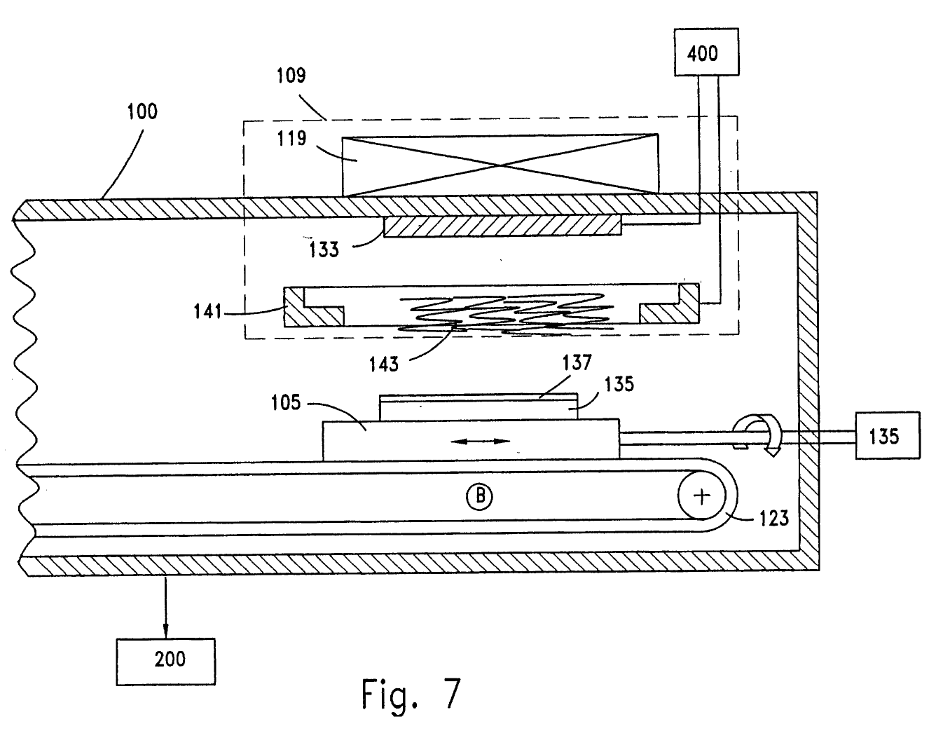

Referring to FIG. 7, the sputtering assembly (109) comprises a magnetic block (119) aligned with respect to a target cathode (133). During operation of the apparatus (100), in particular the sputtering step of the process, the base layer (135) is aligned with the target cathode (133), and an anode (141) is positioned intermediate the cathode (133) and the base layer (135). A DC power source may be use to operate the sputtering assembly (109), wherein the anode (141) and the cathode (133) are operatively connected to a positive and negative terminals, respectively, of a suitable DC power source. Alternatively, the sputtering assembly may be powered by means of a radio frequency induction source, in which case a separate and independent anode (141) may not be required. The target cathode (133) is made from, or at least comprises, the metal from which it is desired to form the said thin substantially amorphous layer (137) on the base layer (135), and is typically in the form of a disc, though it may be in any desired shape. Thus, the cathode (133) is preferably made from, or at least comprises a suitable metal having a substantially non-positive electrode potential, and thus may include any one of titanium, zirconium, hafnium, vanadium, niobium, tantalum, chromium, molybdenum or tungsten, and preferably titanium, or any suitable alloy comprising one or more of these metals, for example.

The anode (141) is typically annular, or at least has a shape that circumscribes the periphery of the cathode (133), providing a central aperture for providing communication between the cathode (133) and the base layer (135) when this is positioned in alignment with the cathode target (133) at the sputtering station (B).

Thus, the metal to be sputtered has the potential of cathode (133), and the anode (141) is placed a few centimeters from the cathode (133), between the latter and the substrate, which for the sake of simplicity is herein taken to be a base layer (135) of an implantable device of any suitable shape. The anode (141) typically has an earth potential. The magnet block (119) is located above the cathode (133), so that on application of a negative potential to the cathode (133), a cross-field space in which the electric and magnetic fields intersect, is created near the cathode (133) in which a cloud of plasma (143) is formed and concentrated. (The plasma cloud (143) comprises argon ions, originally provided as argon gas from the argon supply source (300), after the working chamber (113) has been suitably evacuated of air therein.) Cations in the plasma cloud (143) are accelerated towards the cathode-target (133) and bombard its surface to dislodge and sputter the metal atoms therefrom. The metal species leaving the surface of the cathode-target (133) are then deposited as a thin metal layer (or film) (137) on the base layer (135). Localization of the electrons near the cathode-target, resulting from this arrangement, prevents electron bombardment of the base layer (135), arid accordingly its temperature is kept low and the number of radiation defects of the deposited metal layer is diminished. Further, the adhesion to the base layer (135) of the metal layer formed by this method is significantly higher than of that achieved using other methods, for example, vacuum evaporation method. This is because of the high energy of the sputtered metal species, which is between about 5 to about 10 electron-volt, compared with less than about 1 electron-volt in case of vacuum evaporation method.

The magnetron apparatus (100) may thus be used for forming a thin, substantially non-continuous metal layer on a suitable substrate (135), as follows, the base layer (135) typically being made from a suitable synthetic material chosen from polyurethane, including different co-polymers thereof and polyurethane-derived materials, polytetrafluoroethylene, polyethylene glycol terephthalate, or expanded microporous polytetrafluoroethylene, and other suitable polymeric materials.

A base layer (135) of suitable size, shape and thickness is placed in an ultrasonic bath containing a low residue detergent solution in distilled water for ultrasonic cleansing. This is followed by rinsing by freshly distilled water, and the base layer (135) is then dried in a desicator at ambient temperature. Then the base layer (135) is then placed in the substrate holder (105), and inserted into the working chamber (113) via the air-lock (103). The working chamber (113) is then evacuated by means of the evacuation system (200) to a residual pressure of about 0.1 Pa, and an atmosphere of argon is created in the working chamber (113) by means of the argon supply source (300). The base layer (135) is then moved into position at station (A), i.e., the ionic etching position (A), in opposed relationship to the ion source (115) by correspondingly displacing the substrate holder (105) by means of the conveyor system (123). Ionic etching of the base layer (135), or at least the upper or outer surface thereof is then performed at the residual pressure of 0.1-1.0 Pa in order to create an unadulterated surface on the base layer (135), for subsequently enabling the metal layer to be formed with a high degree of adhesion to the base layer (135). Then the base layer (135) is moved into the sputtering position (B), i.e. opposite the target-cathode (133), by correspondingly displacing the substrate holder (105) by means of the conveyor system (123). Rotational means (135) may then be brought into engagement with the substrate holder (105) to turn the base layer (135) about a suitable axis so as to enable other parts of the base layer (135) to be exposed to the sputtering, particularly where the base layer (135) is in tubular form or round. Alternatively, the substrate holder (105) itself may be adapted to enable the base layer (135) to be rotated about any suitable axis. Additionally or alternatively, the conveyor system (123), or indeed any other suitable system incorporated in the apparatus (100), may provide a reciprocating movement to the substrate holder (105) to increase the extent of exposure of the base layer (135) to the sputtering, particularly where the base layer (135) is in sheet form.

A glow discharge is then initiated in the working chamber (113) above the target-cathode (133), and cations of argon are accelerated towards the target-cathode 133 and bombard its surface. As a result the metal species leaving the surface of the cathode-target (133) are deposited as a thin metal layer (or film) on the base layer (135). This step of the sputtering process is typically performed with argon plasma at pressures ranging from about 0.3 Pa to about 1.0 Pa. Further increase of pressure typically worsens the adhesion to the base layer (135), while further lowering of pressure generally results in substantial crystallinity of the produced metal layer. The optimal value of the power density of sputtering typically lies between about 4.0 Watt/cm² to about 6.0 Watt/cm². Increasing further the power density generally results in overheating and distortion of the base layer (135), and hence loss of elasticity, while decreasing the power density to below about 4.0 Watts/cm² results in considerable decrease of flux density of the metal species and, therefore, of efficiency of the sputtering process. The sputtering step is typically carried out at a potential from approximately 200 Volt to approximately 500 Volt between the anode (141) and cathode (133).

The average thickness of the substantially amorphous or quasi amorphous, and preferably non-continuous, metal layer, applied to the surface of the base layer (135) is typically between about 50 nanometers to about 300 nanometers, with respectively 50% to 95% percent of the surface of the base layer (135) actually being covered by metal If the average thickness is less than about 50 nanometers formation of the electrostatic charges on the surface of the base layer (135) may not be completely avoided, which may in turn cause formation of thrombosis. Total continuity of the metal layer is typically achieved when its average thickness exceeds 300 nanometers. In this case the elasticity of the base layer (135) is significantly diminished, and the metal layer becomes prone to cracking upon stretching or bending of the base layer (135). The non-continuous structure of the metal layer (at the micro-scale) allows the mechanical tensions to relax and, therefore, enhances adhesiveness and robustness of the metal layer.

Typically, at least part of the resulting thin substantially amorphous and preferably non-continuous metal layer is then oxidised, either by exposing said metal layer to the atmosphere or to a bloodstream.

The said base layer (135) may optionally be provided in the form of a sheet particularly adapted for providing a vascular patch, wherein said substantially non-continuous metal layer is provided on the bloodstream facing layer of said sheet. Alternatively, the said base layer may be provided in the form of at least one component of a suitable artificial heart valve, wherein said thin substantially non-continuous metal layer is provided on the bloodstream facing layers of said at least one component of said suitable artificial heart valve. Alternatively, the base layer (135) may be provided in the form of at least one component of a suitable artificial heart assembly, wherein said thin substantially amorphous and preferably non-continuous metal layer is provided on the bloodstream facing layers of said at least one component of said suitable artificial heart assembly.

The magnetic sputtering method according to the present invention may also be used successfully for prosthesis material in the tubular form, such as in the case of a vascular shunt or graft, in which it is required to apply a thin substantially amorphous and preferably non-continuous metal film to the inner facing cylindrical surface thereof. The method according to the present invention allows for simplification of the technological process required for this purpose, the internal surface of a vascular shunt or graft being first inverted inside out so as to expose the inner cylindrical surface outwardly. Then the exposed surface is subjected to metal sputtering, and, finally, the vascular graft is inverted once again back to its original condition, in such a manner that the thin protective metal layer is now situated in the internally-facing surface of the tubular body of a vascular shunt or graft, where it is exposed to the recipient's bloodstream.

Whatever the form of the base layer (135), the combined effects of the electric and magnetic fields in the working chamber (113) produces spiral trajectory and lengthening of the path of motion of electrons with corresponding enhancement of the efficiency of ionization. Thus, a cloud of dense low impedance plasma is formed, and sputtering occurs at the potential of 200-500 Volts. As a result, the efficiency of the system is considerably enhanced, and heating of the base layer (135) is diminished, while the non-thrombogenic properties of the metal layer or coating formed thereby is improved.

### EXAMPLES

The following experiments were conducted on two pairs of dogs, at different times, in which the operations were performed without the use of heparin, and in which the dogs received no anti-platelet treatment. One pair of dogs was subjected to a first operation, and the second pair of dogs to a different second operation,.

In the first operation, one 5 cm length, 8-mm internal diameter graft was used to replace a 3 cm long section from the abdominal aorta of each dog.

Thus, for each dog in turn, under intravenous general anesthesia the abdomen was opened through a mid-line incision. The abdominal aorta was separated from below the renal arteries up to the aortic bifurcation. After clamping, a 3-cm part of aorta was resected and 8-mm internal diameter, 5 cm long titanium coated graft was sutured in end-to-end fashion with two 5-0 Prolen sutures on each end. After haemostasis with Surgi-Cel, the abdomen was closed in 2-layer fascial sutures using 2-0 Vycryl. The skin incision was closed with a 3-0 Vycryl single suture.

One dog in this pair was sacrificed after about 1 month, and the second dog after about 3 months. The grafts were excised from each of the dogs after sacrifice. All the grafts were found to be clean from thrombus.

In the second operation, two 5 cm length, 4-mm internal diameter grafts were used to replace two carotid arteries (one at each side of the neck) of each dog.

Thus, for each dog, under intravenous general anesthesia, a 10-cm mid-line skin incision was made above the tracheal cartilage. Using blunt preparation between trachea and long cervical muscle, a 7-8 cm of length of the carotid artery was separated. Carotid artery was occluded with two microvascular clamps (Heifitz) and 4-cm part of the artery was resected. The 5-cm Titanium-coated 4-mm PTFE graft was sutured in end-to-end fashion using two 6-0 Prolen sutures on each end. After completion of anastomoses the graft was covered with cervical muscle. The same procedure was performed on the other side of the neck. The skin incision was closed with a 3-0 Vycryl single suture.

One dog in this pair was sacrificed after about 1 month, and the second dog after about 3 months. The grafts were excised from each of the dogs after sacrifice. All the grafts were found to be clean from thrombus.

Results

1. Dogs Sacrificed After One Month

Gross Examination

The graft surface is smooth, without signs of thrombosis or fibrin deposits in the areas of sutures between the graft and the vessel wall a mild thickening of the vessel wall and only a few thin fragments of fibrin were seen.

Microscopic Examination

The surface of the graft area is smooth and regularly covered by a thin layer of an amorphic material 0 1-0.2 mm thick. No signs of thrombosis or platelet adhesion were found. In the area of sutures there is a slight protrusion of the vessel wall and an inflammatory infiltrate in the wall of the vessel. The infiltrate contains lymphocytes, plasma cells and a few granulocytes, thickened blood vessels (vasa vasorum) are seen in the vessel wall. No thrombosis was seen. There was one small area on the wall of the grafts where a small fragment of fibrin was seen in the lumen without attachment to the graft inner surface.

2. Does Sacrificed After Three Months

Gross Examination

The graft surface is smooth, no signs of thrombosis were seen. In the area of sutures between the graft and the vessel wall there was a thickening of the vessel without signs of thrombosis.

Microscopic Examination

The surface of the graft is smooth. No signs of thrombosis or platelet adhesion were seen. The area of sutures shows thickening of the wall and areas of fibrosis. A mild inflammatory infiltrate in the media of the vessel was seen. No thrombosis was found.

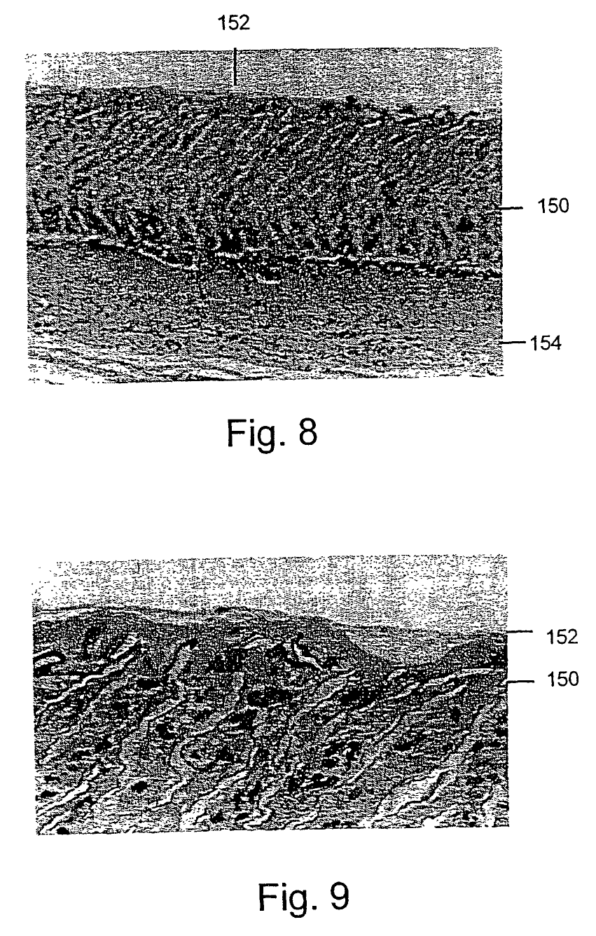

FIGS. 8 and 9 show typical microscope images at about 80 and about 250 magnifications, respectively of the titanium coated graft after the experiments in which the dogs were sacrificed after 3 months. No significant differences were found between the results obtained with the dog that had the first operation and with the dog that had the second operation was completed. Referring to these figures, teflon graft (150) can be seen as having a fibrous texture, having a thin non-continuous titanium layer (152) on the bloodstream facing surface of the graft. The other side of the graft (150) abuts the coarctate biological tissue (154) of the dog. As is clear from these figures, the bloodstream facing surfaces of the graft covered by the titanium layer (152) are substantially free of adsorbed platelets and blood elements of the recipient dog's blood, which would normally contribute to die formation of thromboses These positive results provide evidence that the prosthetic material comprising a base layer and a thin, substantially non-continuous layer of a suitable metal according to the present invention provides excellent non-thrombogenic properties.

Thus, the present invention also relates to a method for replacing a vascular tissue with a non-thrombogenic implant comprising the steps of:

surgically removing said vascular tissue;

surgically implanting a suitable non-thrombogenic implantable device as described hereinbefore, mutatis mutandis.

Further, the present invention is also directed to a method for repairing a vascular tissue with a non-thrombogenic implant comprising the steps of:

surgically preparing a damaged part of said vascular tissue to receive an implant;

surgically implanting a suitable non-thrombogenic implantable device as described hereinbefore, mutatis mutandis, on said damaged part of said vascular tissue.

While in the foregoing description describes in detail only a few specific embodiments of the invention, it will be understood by those skilled in the art that the invention is not limited thereto and that other variations in form and details may be possible without departing from the scope and spirit of the invention herein disclosed or exceeding the scope of the claims.

1. A non-thrombogenic prosthetic material for the vascular system of the body having, at least one bloodstream facing surface, comprising a base layer made from a suitable material, and a thin substantially amorphous or at least partially amorphous layer of a suitable metal covering at least part of said base layer, said metal layer comprising said at least one bloodstream facing surface, wherein said metal layer is made from a suitable metal such as to provide a substantially non-positive electrode potential with respect to a bloodstream in contact with said metal layer.

2. A non-thrombogenic prosthetic material as claimed in claim 1, wherein said metal layer is substantially non-continuous.

3. A non-thrombogenic prosthetic material as claimed in claim 1, wherein said metal layer is made from a metal having a substantially non-positive standard electrode potential.

4. A non-thrombogenic prosthetic material as claimed in claim 1, wherein said metal layer comprises a thickness which may vary from between about 0 nm and about 400 nm.

5. A non-thrombogenic prosthetic material as claimed in claim 1, wherein said metal layer comprises an average thickness of between 50 nm to between about 300 nm., and preferably about 200 nm.

6. A non-thrombogenic prosthetic material as claimed in claim 1, wherein said metal layer is made from any one of titanium, zirconium, hafnium, vanadium, niobium, tantalum, chromium, molybdenum or tungsten, or any suitable alloy comprising at least one of titanium, zirconium, hafnium, vanadium, niobium, tantalum, chromium, molybdenum or tungsten.

7. A non-thrombogenic prosthetic material as claimed in claim 6, wherein said metal layer comprises an oxide of any one of titanium, zirconium, hafnium, vanadium, niobium, tantalum, chromium, molybdenum or tungsten.

8. A non-thrombogenic prosthetic material as claimed in claim 1, said material further comprising at least one body-tissue facing surface adapted for implantation in a body tissue.

9. A non-thrombogenic prosthetic material as claimed in claim 8, wherein said base layer is made from a substantially homogenous suitable synthetic material.

10. A non-thrombogenic prosthetic material as claimed in claim 8, wherein said base layer is made from a synthetic material chosen from polyurethane, including different co-polymers thereof and polyurethane-derived materials, polytetrafluoroethylene, polyethylene glycol terephthalate, or expanded microporous polytetrafluoroethylene, and other suitable polymeric materials.

11. A non-thrombogenic prosthetic material as claimed in 8, wherein said material is in the form of a device adapted for implantation in the body.

12. A non-thrombogenic prosthetic material as claimed in 11, wherein said device is in the form of a patch adapted for grafting onto a predetermined part of the vascular system.

13. A non-thrombogenic prosthetic material as claimed in claim 11, wherein said device is in the form of a prosthesis adapted for suitable implantation in the vascular system of the body.

14. A non-thrombogenic prosthetic material as claimed in 13, wherein said prosthesis is in the form of a vascular graft or shunt.

15. A non-thrombogenic prosthetic material as claimed in 14, wherein said prosthesis comprises said material in tubular form, having an inner substantially cylindrical bloodstream facing surface, said metal layer comprising said bloodstream facing surface

16. A non-thrombogenic prosthetic material as claimed in 14, wherein said prosthesis comprises said material in the form of at least one component of a suitable artificial heart valve, said at least one component thereof having at least one bloodstream facing surface, said metal layer comprising said at least one bloodstream facing surface.

17. A non-thrombogenic prosthetic material as claimed in 14, wherein said prosthesis comprises said material in the form of at least one component of a suitable artificial heart assembly, said at least one component having at least one bloodstream facing surface, said thin metal layer comprising said at least one bloodstream facing surface.

18. A non-thrombogenic prosthetic material as claimed in any one of claims 1 to 17, wherein said base layer is covered by said metal layer by means of a magnetron sputtering based procedure.

19. A non-thrombogenic implantable device for the vascular system of the body having at least one bloodstream facing surface, comprising a base layer made from a suitable material, and a thin substantially amorphous or at least partially amorphous layer of a suitable metal covering at least part of said base layer, said metal layer comprising said at least one bloodstream facing surface, wherein said metal layer is made from a suitable metal such as to provide a substantially non-positive electrode potential with respect to a bloodstream in contact with said metal layer.

20. A substantially non-thrombogenic implantable device as claimed in claim 19, wherein said device is in the form of a patch adapted for grafting onto a predetermined part of the vascular system.

21. A non-thrombogenic implantable device as claimed in claim 19, wherein said device is in the form of a prosthesis adapted for suitable implantation in the vascular system of the body.

22. A non-thrombogenic implantable device as claimed in claim 21, wherein said prosthesis is in the form of a vascular graft or shunt.

23. A non-thrombogenic implantable device as claimed in claim 22, wherein said prosthesis comprises said material in tubular form, having an inner substantially cylindrical bloodstream facing surface, said metal layer comprising said bloodstream facing surface.

24. A non-thrombogenic implantable device as claimed in claim 22, wherein said prosthesis comprises said material in the form of at least one component of a suitable artificial heart valve, said at least one component thereof having at least one bloodstream facing surface, said thin metal layer comprising said at least one bloodstream facing surface.

25. A non-thrombogenic implantable device as claimed in claim 22, wherein said prosthesis comprises said material in the form of at least one component of a suitable artificial heart assembly, said at least one component having at least one bloodstream facing surface, said thin metal layer comprising said at least one bloodstream facing surface.

26. A non-thrombogenic implantable device as claimed in any one of claims 19 to 25, wherein said metal layer is substantially non-continuous.

27. A non-thrombogenic implantable device as claimed in any one of claims 19 to 25, wherein said metal layer is made from a metal having a substantially non-positive standard electrode potential.

28. A non-thrombogenic implantable device as claimed in any one of claims 19 to 25, wherein said metal layer comprises a thickness which may vary from between about 0 nm and about 400 nm

29. A non-thrombogenic implantable device as claimed in any one of claims 19 to 25, wherein said metal layer comprises an average thickness of between 50 nm to between about 300 nm., and preferably about 200 nm.

30. A non-thrombogenic implantable device as claimed in any one of claims 19 to 25, wherein said metal layer is made from any one of titanium, zirconium, hafnium, vanadium, niobium, tantalum, chromium, molybdenum or tungsten, or any suitable alloy comprising at least one of titanium, zirconium, hafnium, vanadium, niobium, tantalum, chromium, molybdenum or tungsten.

31. A non-thrombogenic implantable device as claimed in claim 30, wherein said metal layer comprises an oxide of any one of titanium, zirconium, hafnium, vanadium, niobium, tantalum, chromium, molybdenum or tungsten.

32. A non-thrombogenic implantable device as claimed in any one of claims 19 to 25, said material further comprising at least one body-tissue facing surface adapted for implantation in a body tissue.

33. A non-thrombogenic implantable device as claimed in claim 32, wherein said base layer is made from a substantially homogenous suitable synthetic material.

34. A non-thrombogenic implantable device as claimed in claim 33, wherein said base layer is made from a synthetic material chosen from polyurethane, including different co-polymers thereof and polyurethane-derived materials, polytetrafluoroethylene, polyethylene glycol terephthalate, or expanded microporous polytetrafluoroethylene, and other suitable polymeric materials.

35. A non-thrombogenic implantable device as claimed in any one of claims 19 to 25, wherein said base layer is covered by said thin substantially amorphous metal layer by means of a magnetron sputtering based procedure.

36. Method for providing a non-thrombogenic material for the vascular system of the body having at least one bloodstream facing surface, comprising covering at least a portion of a base layer made from a suitable material with a thin substantially amorphous or at least partially amorphous layer of a suitable metal, said at least one bloodstream facing surface of the non-thrombogenic material being comprised on said thin metal layer, wherein said metal layer is made from a suitable metal such as to provide a substantially non-positive electrode potential with respect to a bloodstream in contact with said metal layer.

37. Method as claimed in claim 36, wherein said metal layer is applied non-continuously over said base layer.

38. Method as claimed in claim 36, wherein base layer is covered by said metal layer by means of a magnetron sputtering based procedure.

39. Method as claimed in claim 38, wherein said magnetron sputtering based procedure comprises the following steps:
(a) providing said base layer made from a suitable material and in a suitable form;
(b) placing said base layer in a vacuum chamber comprising suitable magnetron sputtering means;
(c) providing a target made from said suitable metal in said vacuum chamber;
(d) evacuating the chamber to a residual pressure;
(e) providing an atmosphere of plasma forming gas in said vacuum chamber;
(f) initiating a suitable electrical glow discharge in said vacuum chamber to provide plasma ions from said plasma forming gas directed at said metal target;
(g) sputtering metal from said metal target onto said base layer responsive to interaction of said plasma ions onto said metal target whereby to cover said base layer with a thin substantially non-continuous layer of said metal.

40. Method as claimed in claim 39, further comprising the following step between steps (a) and (b):
(h) cleansing said base layer using any suitable cleansing method;

41. Method as claimed in claim 40, wherein said cleansing method is an ultrasonic-based cleansing method.

42. Method as claimed in claim 39, further comprising the following step between steps (e) and (f):
(i) ionically etching at least one outer surface of said base layer;

43. Method as claimed in claim 42, wherein said plasma forming gas is argon.

44. Method as claimed in claim 43, wherein said ionic etching step is performed at a pressure of between about 0.1 Pa to about 1.0 Pa, and preferably between about 0.3 Pa to about 1.0 Pa.

45. Method as claimed in claim 44, wherein a power density associated with said magnetically sputtering step is between about 4.0 W/cm² to about 6.0 W/cm².

46. Method as claimed in claim 38, wherein a potential associated with said magnetically sputtering step is between about 200V and between 500V.

47. Method as claimed in claim 41, wherein said magnetically sputtering step is performed until an average thickness associated with said substantially non-continuous metal layer reaches between about 50 nm to between about 300 nm.

48. Method as claimed in claim 38, wherein said base layer is made from a suitable synthetic material

49. Method as claimed in claim 38, wherein said base layer is made from a suitable synthetic material chosen from polyurethane, including different co-polymers thereof and polyurethane-derived materials, polytetrafluoroethylene, polyethylene glycol terephthalate, or expanded microporous polytetrafluoroethylene, and other suitable polymeric materials.

50. Method as claimed in claim 38, wherein said metal target comprises a metal chosen from among any one of titanium, zirconium, hafnium, vanadium, niobium, tantalum, chromium, molybdenum or tungsten, or an alloy comprising at least one of titanium, zirconium, hafnium, vanadium, niobium, tantalum, chromium, molybdenum or tungsten.

51. Method as claimed in claim 50, further comprising the step of oxidising at least a portion of said substantially amorphous metal layer.

52. Method as claimed in claim 50, wherein said oxidising step comprises exposing said metal layer to the atmosphere.

53. Method as claimed in claim 50, wherein said oxidising step comprises exposing said metal layer to a bloodstream.

54. Method as claimed in claim 38, wherein said base layer is provided in the form of a sheet particularly adapted for providing a vascular patch, wherein said metal layer is provided on the bloodstream facing layer of said sheet.

55. Method as claimed in claim 38, wherein said base layer is provided in the form of a tube, said method further comprising the steps:
(j) inverting the tube inside out so that the inner cylindrical surface is now outermost;
(k) re-inverting the tube so that the said inner cylindrical surface in innermost again;
wherein step (j) is performed before step (b), and step (k) is performed after step (g), whereby said metal layer is provided on said inner cylindrical surface of said tube.

56. Method as claimed in claim 38, wherein said base layer is provided in the form of at least one component of a suitable artificial heart valve, wherein said thin metal layer is provided on the bloodstream facing layers of said at least one component of said suitable artificial heart valve.

57. Method as claimed in claim 38, wherein said base layer is provided in the form of at least one component of a suitable artificial heart assembly, wherein said thin metal layer is provided on the bloodstream facing layers of said at least one component of said suitable artificial heart assembly.

58. A non-thrombogenic prosthetic material for the vascular system of the body made by the method as claimed in any one of claims 36 to 57.

59. A non-thrombogenic implant for the vascular system of the body made by the method as claimed in any one of claims 36 to 57.

60. A method for replacing a vascular tissue with a non-thrombogenic implant comprising the steps of:
(a) surgically removing said vascular tissue;
(b) surgically implanting a suitable non-thrombogenic implantable device according to any one of claims 19 to 35.

61. A method for repairing a vascular tissue with a non-thrombogenic implant comprising the steps of:
(c) surgically preparing a damaged part of said vascular tissue to receive an implant;
(d) surgically implanting a suitable non-thrombogenic implantable device according to any one of claims 19 to 35 on said damaged part of said vascular tissue.
